(19) United States
(12) Patent Application Publication
(10) Pub. No.: US 2026/0248652 A1
(43) Pub. Date: Aug. 27, 2026

(54) SNOW GOGGLES DEVICE AND MANUFACTURING METHOD THEREOF HAVING AN ACTIVELY DEFROSTING THIH FILM

(71) Applicant: FORESIGHT OPTICAL LTD., Tainan (TW)

(72) Inventors: TIEN-SHU WU, Tainan (TW); YEN-TING WU, Tainan (TW)

(21) Appl. No.: 19/187,808

(22) Filed: Apr. 23, 2025

(30) Foreign Application Priority Data
Feb. 27, 2025 (TW) 114107486

(51) Int. Cl. A61F 9/02; G02C 11/08

(52) U.S. Cl. CPC A61F 9/029; G02C 11/08

(57) ABSTRACT

A manufacturing method of a snow goggles device includes: providing an optical substrate layer on a snow goggles frame; providing the optical substrate layer with a first surface and a second surface, and providing at least one transparent photo-thermal conversion film on the first surface, the second surface or therebetween; the transparent photo-thermal conversion film absorbing a light ray or a light beam to convert into heat energy; and utilizing the heat energy generated the transparent photo-thermal conversion film to heat the optical substrate layer.

### BACKGROUND OF THE INVENTION

### 1. Field of the Invention

The present invention relates to a snow goggles device and manufacturing method thereof having an actively defrosting thin film. Particularly, the present invention relates to the snow goggles device and manufacturing method thereof having an actively defrosting thin film on a surface of glasses. More particularly, the present invention relates to the snow goggles device and manufacturing method thereof having an actively defrosting thin film directly heating the surface of glasses.

### 2. Description of the Related Art

Chinese Utility-Model Patent Publication No. CN-211985900, entitled “Goggles,” discloses a goggles device, including a goggles frame, a desiccant compartment, a transparent heating glasses plate and a power supply which has a battery socket and a battery unit mounted therein.

As described above, the goggles frame has an inner wall on which to mount the desiccant compartment. Provided between the goggles frame and the desiccant compartment is a socket-plug connection arc support structure which is a mutually coupled and detachable arc support structure.

As described above, the goggles frame is assembled with the transparent heating glasses plate which is provided with a sandwiched film layer and a heating wire formed therewith. The heating wire heating wire electrically connects with the power supply which has a control switch.

As described above, the goggles frame further includes an exterior power supply connection hole which is provided with a USB interface. The goggles frame further includes a thermal sensor which is electrically connected with a thermal adjustment knob.

However, the goggles frame has the transparent heating glasses plate which is directly heated by the heating wire formed in the sandwiched film layer. Disadvantageously, the heating wire causes interference of vision through the transparent heating glasses plate such that there is a need of improving the design of transparent heating glasses plates of goggles device.

Another Canadian Patent Application Publication No. Ca-2,327,006, entitled “Anti-frost goggles and method for manufacturing the same,” discloses an anti-frost goggles device and a manufacturing method thereof. The anti-frost goggles device has a goggles body performed as a frame.

As described above, the goggles body has an inner surface on which to provide a heating element for heating thereon. The heating element further includes a pair of electrodes also provided at the inner surface of the goggle body where has upper and lower portions to provide each of electrodes.

As described above, the pair of electrodes electrically connects with a terminal via a pair of power supply lines and a pair of external wires. The terminal is connected between power supply means and the power supply lines via the external electric wires. A power switch is coupled between the terminal and the power supply means.

However, the heating element is only provided to heat the inner surface of the goggles body. Disadvantageously, the heating element cannot directly heat a glasses lens of the anti-frost goggles device such that there is a need of improving the design of heating for the anti-frost goggles device.

Another Taiwanese Patent Publication No. TW-336691, entitled “Edge coated glass polarized lens,” discloses a glasses polarization lens device formed with an adhesive-coated side edge. The glasses polarization lens device includes a first glasses layer, a second glasses layer, a polarization layer and an adhesive-coated side edge layer.

As described above, the first glasses layer has a first optical surface while the second glasses layer has a second optical surface opposite to the first optical surface. The polarization layer is provided between the first glasses layer and the second glasses layer. The adhesive-coated side edge layer is formed on a peripheral side edge connected between the first glasses layer and the second glasses layer.

As described above, the adhesive-coated side edge layer is formed by polymerization chain reaction of free radicals of UV adhesive material exposing or curing in UV light. The UV adhesive material includes aliphatic polyesterpolyol based urethane methacrylate, a reactive diluent, an adhesion promoter and a photo-initiator. The aliphatic polyesterpolyol based urethane methacrylate is selectively made of aliphatic polyester polyol, methyl methacrylate (MMA) and di-isocyanate and has an average mass ranging between 800 and 4,000.

As described above, the reactive diluent is selected from methyl methacrylate monomer and the adhesion promoter is made of methacrylate-functional silane, amino methacrylate-functional silane, vinyl-functional silane and combinations thereof. The photo-initiator is made of alpha-hydroxy ketone, alpha-amino ketone, benzil dimethyl ketal, benzophenone, phosphine oxide and combinations thereof.

Another U.S. Pat. No. 4,076,863, entitled “Process for cutting and edging chemically pre-strengthened finished uncut lens blanks without loss of impact resistance,” also discloses an edging chemically pre-strengthened finished uncut lens blank which has a side peripheral surface and a side coated layer formed thereon.

As described above, the side coated layer is selectively made of epoxy resin, alkyd resin, polyurethane resin, unsaturated polyester resin, acrylic resin, cyanoacrylate and combinations thereof.

Another U.S. Pat. No. 5,220,358, entitled “Edge coating for laminated lenses,” also discloses an edge-coated, laminated lens device. The edge-coated, laminated lens device has a side peripheral surface and a side coated layer formed thereon.

As described above, the side coated layer is selected from a dimethyl silicone-coated layer. The side coated layer comprises a dimethyl silicone coblocked with vinyl with crosslinking therein being provided through a platinum-catalyzed addition reaction.

Another U.S. Pat. No. 6,860,600, entitled “Edge coated ophthalmic lenses,” also discloses an edge coated ophthalmic lens device. The edge coated ophthalmic lens device has a side peripheral surface and a side coated layer formed thereon.

As described above, the side coated layer of peripheral edge is coated with an edge coating which strongly bonds to the peripheral edge but that only weakly bonds to an optical surface, and the edge coating is durable. A composition of the side coated layer is selected from UV curable acrylates.

However, there is a need of improving the conventional snow goggles device or structure for providing an actively defrosting snow goggles device and manufacturing method thereof. The above-mentioned patents and patent application publications are incorporated herein by reference for purposes including, but not limited to, indicating the background of the present invention and illustrating the situation of the art.

### SUMMARY OF THE INVENTION

The primary objective of this invention is to provide a snow goggles device and manufacturing method thereof having an actively defrosting thin film. An optical substrate layer provided on a snow goggles frame has a f irst surface and a second surface, with providing at least one transparent photo-thermal conversion film on the first surface, the second surface or therebetween. In use, the transparent photo-thermal conversion film can actively absorb a light ray or beam to convert into heat energy which can be utilized to heat the optical substrate layer. Advantageously, the snow goggles device of the present invention is successful in preventing or avoiding the optical substrate layer turning into a frozen state which will reduce visibility.

The snow goggles device in accordance with an aspect of the present invention includes:
an optical substrate layer provided with a first surface and a second surface;
a snow goggles frame provided with a predetermined surface, with providing the optical substrate layer on the predetermined surface of the snow goggles frame; and
at least one transparent photo-thermal conversion film provided on the first surface, the second surface or therebetween;
wherein the transparent photo-thermal conversion film can actively absorb a light ray or a light beam to convert into heat energy which can be utilized to heat the optical substrate layer for preventing the optical substrate layer turning into a frozen state.

In a separate aspect of the present invention, the transparent photo-thermal conversion film formed on the optical substrate layer further electrically connects with at least one solar cell unit.

In a further separate aspect of the present invention, the transparent photo-thermal conversion film is integrally formed on the snow goggles frame, with the transparent photo-thermal conversion film further electrically connects with at least one solar cell unit.

In yet a further separate aspect of the present invention, the transparent photo-thermal conversion film is made of a photo-thermal conversion material or a mixture of photo-thermal conversion material and thermochromic dye material.

In yet a further separate aspect of the present invention, the photo-thermal conversion material is selected from an infrared-thermal conversion material.

In yet a further separate aspect of the present invention, the infrared-thermal conversion material includes a plurality of tungsten oxide micro-particles, a plurality of composite tungsten oxide micro-particles, a plurality of titanium dioxide micro-particles, a plurality of composite titanium dioxide micro-particles, a plurality of titanium dioxide/antimony-doped tin oxide (TiO₂@ATO) core-shell micro-particles and combinations thereof.

In yet a further separate aspect of the present invention, the transparent photo-thermal conversion film is further formed with at least one hard coating layer.

The manufacturing method of the snow goggles device in accordance with another aspect of the present invention includes:
providing an optical substrate layer on a snow goggles frame;
providing the optical substrate layer with a first surface and a second surface, and providing at least one transparent photo-thermal conversion film on the first surface, the second surface or therebetween;
the transparent photo-thermal conversion film actively absorbing a light ray or a light beam to convert into heat energy; and
utilizing the heat energy generated from the transparent photo-thermal conversion film to heat the optical substrate layer for preventing the optical substrate layer turning into a frozen state.

In a separate aspect of the present invention, the transparent photo-thermal conversion film formed on the optical substrate layer further electrically connects with at least one solar cell unit.

In a further separate aspect of the present invention, the transparent photo-thermal conversion film is integrally formed on the snow goggles frame, with the transparent photo-thermal conversion film further electrically connects with at least one solar cell unit.

In yet a further separate aspect of the present invention, the transparent photo-thermal conversion film is made of a photo-thermal conversion material or a mixture of photo-thermal conversion material and thermochromic dye material.

In yet a further separate aspect of the present invention, the photo-thermal conversion material is selected from an infrared-thermal conversion material which is selectively added in the snow goggles frame, the optical substrate layer or both.

In yet a further separate aspect of the present invention, the infrared-thermal conversion material includes a plurality of tungsten oxide micro-particles, a plurality of composite tungsten oxide micro-particles, a plurality of titanium dioxide micro-particles, a plurality of composite titanium dioxide micro-particles, a plurality of titanium dioxide/antimony-doped tin oxide (TiO₂@ATO) core-shell micro-particles and combinations thereof.

In yet a further separate aspect of the present invention, the transparent photo-thermal conversion film is further formed with at least one hard coating layer.

Further scope of the applicability of the present invention will become apparent from the detailed description given hereinafter. However, it should be understood that the detailed description and specific examples, while indicating preferred embodiments of the invention, are given by way of illustration only, since various will become apparent to those skilled in the art from this detailed description.

### BRIEF DESCRIPTION OF THE DRAWINGS

The present invention will become more fully understood from the detailed description given hereinbelow and the accompanying drawings which are given by way of illustration only, and thus are not limitative of the present invention, and wherein:

FIG. 1 is a block diagram of a snow goggles device having an actively defrosting thin film in accordance with a preferred embodiment of the present invention.

FIG. 2 is a schematic front view of a snow goggles device having an actively defrosting thin film in accordance with a first preferred embodiment of the present invention.

FIG. 3 is a schematic side view of a snow goggles device having the actively defrosting thin film in accordance with the first preferred embodiment of the present invention.

FIG. 4 is a flow chart of a manufacturing method of snow goggles device in accordance with a preferred embodiment of the present invention.

FIG. 5 is a block diagram of a snow goggles device having an actively defrosting thin film in accordance with another preferred embodiment of the present invention.

FIG. 6 is a schematic front view of a snow goggles device having an actively defrosting thin film in accordance with a second preferred embodiment of the present invention.

FIG. 7 is a schematic side view of a snow goggles device having the actively defrosting thin film in accordance with the second preferred embodiment of the present invention.

FIG. 8 is a schematic front view of a snow goggles device having an actively defrosting thin film in accordance with a third preferred embodiment of the present invention.

FIG. 9 is a schematic side view of a snow goggles device having the actively defrosting thin film in accordance with the fourth preferred embodiment of the present invention.

### DETAILED DESCRIPTION OF THE INVENTION

It is noted that a snow goggles device and manufacturing method thereof having an actively defrosting thin film in accordance with the preferred embodiment of the present invention can be applicable to various goggles devices, including various industrial goggle devices, various ski or snow goggle devices, various gliding goggle devices, various cycling goggle devices, various motorcycle-riding goggle devices, or other outdoor or indoor goggle devices, which are not limitative of the present invention.

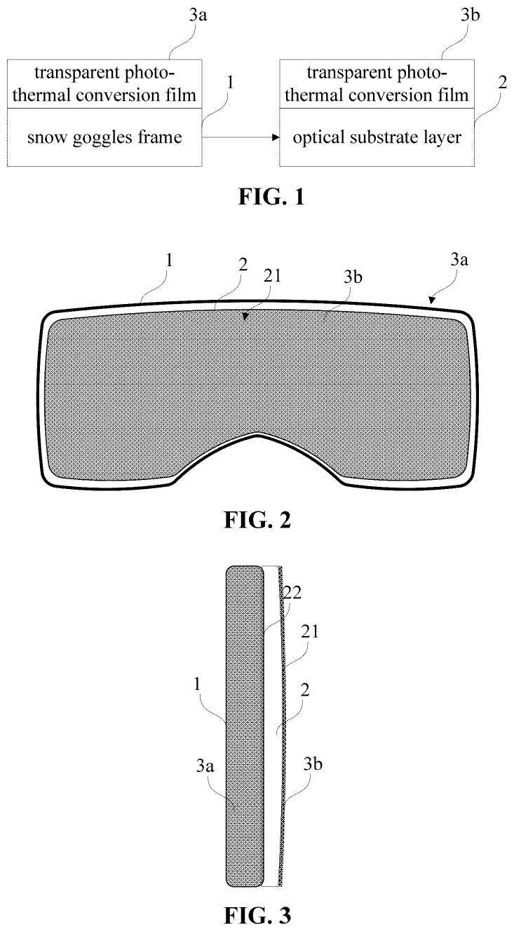

FIG. 1 shows a block diagram of a snow goggles device having an actively defrosting thin film in accordance with a preferred embodiment of the present invention. Referring now to FIG. 1, the snow goggles device in accordance with the preferred embodiment of the present invention includes a snow goggles frame 1, an optical substrate layer 2 and a plurality of transparent photo-thermal conversion films (ultra-thin films) 3a, 3b.

FIG. 2 shows a schematic front view of a snow goggles device having an actively defrosting thin film in accordance with a first preferred embodiment of the present invention. FIG. 3 shows a schematic side view of a snow goggles device having the actively defrosting thin film in accordance with the first preferred embodiment of the present invention, corresponding to the snow goggles device shown in FIG. 2.

Referring now to FIGS. 1, 2 and 3, by way of example, the snow goggles frame 1 has a predetermined outline, e.g. outlines of industrial goggles, ski goggles or other usage goggles, a predetermined pattern, a predetermined color or a predetermined strengthen structure.

With continued reference to FIGS. 1, 2 and 3, by way of example, the snow goggles frame 1 is selectively made of a predetermined material to form a predetermined shape, e.g. shapes of industrial goggles, ski goggles or other usage goggles, with selecting the predetermined material from a plastic material, an alloy metal material, a composite material (e.g., carbon fiber) or combinations thereof.

With continued reference to FIGS. 1, 2 and 3, by way of example, the optical substrate layer 2 is formed from a single layer or multiple layers, with the optical substrate layer 2 having a first thickness (i.e. uniform or varying thickness). The optical substrate layer 2 is selected from a glass optical layer, a plastic optical layer, a biodegradable plastic optical layer, a PC (polycarbonate) optical layer, a PMMA (poly(methyl methacrylate)) optical layer, a nylon optical layer, or other similar material layers thereof.

With continued reference to FIGS. 1, 2 and 3, by way of example, the optical substrate layer 2 is selected from a flat lens or a curved lens, with the curved lens selected from a cylindrical lens, a spherical lens, an ellipsoidal lens or other curved lens. The optical substrate layer 2 can also formed from a single-layer lens, a single-layer composite lens, a double-layer lens (i.e., inner-and-outer combined structure), a double-layer hollow lens (i.e., inner-and-outer spaced structure) or other types of lens.

Still referring to FIGS. 1, 2 and 3, by way of example, the optical substrate layer 2 is selected from a polarization thin-film layer, an anti-reflection layer, a photochromic layer, an anti-blue layer, an anti-blue UV layer, an anti-infrared layer, a chroma enhancement layer, other functional thin-film layer (e.g., anti-fog layer or scratch-resistant layer) or combinations thereof.

With continued reference to FIGS. 1, 2 and 3, by way of example, the optical substrate layer 2 has a first surface 21 and a second surface 22, with the first surface 21 performing as a front-side surface (i.e., front outer surface), with the second surface 22 performing as a rear-side surface (i.e., rear inner surface).

With continued reference to FIGS. 1, 2 and 3, by way of example, the snow goggles frame 1 and the optical substrate layer 2 can be selectively made of same materials (e.g., similar materials) or dissimilar materials (e.g., heterogeneous materials) to provide various characteristics of similar or dissimilar materials.

With continued reference to FIGS. 1, 2 and 3, by way of example, the transparent photo-thermal conversion films 3a, 3b as active thermal generating films, gray areas as best shown in FIG. 2, can be integrally formed on a predetermined surface of the snow goggles frame 1, with formed as a transparent photo-thermal conversion ultra-thin film integrally formed on the snow goggles frame 1. The transparent photo-thermal conversion films 3a, 3b can also be integrally formed on one surface of the optical substrate layer 2, with formed as a transparent photo-thermal conversion ultra-thin film integrally formed on the optical substrate layer 2.

Still referring to FIGS. 1, 2 and 3, by way of example, the transparent photo-thermal conversion films 3a, 3b are selectively made of a photo-thermal conversion material or a mixture of photo-thermal conversion material and thermochromic dye material, with the photo-thermal conversion material being selected from an infrared-thermal conversion material. In another preferred embodiment, the infrared-thermal conversion material includes a plurality of tungsten oxide micro or nano-particles, a plurality of composite tungsten oxide micro or nano-particles, a plurality of titanium dioxide micro or nano-particle, a plurality of composite titanium dioxide micro or nano-particles, a plurality of titanium dioxide/antimony-doped tin oxide (TiO₂@ATO) core-shell micro or nano-particless and combinations thereof which are provided in multiple layers of the transparent photo-thermal conversion films 3a, 3b.

With continued reference to FIGS. 1, 2 and 3, by way of example, the photo-thermal conversion materials of the transparent photo-thermal conversion films 3a, 3b of another preferred embodiment can be directly added in the snow goggles frame 1, the optical substrate layer 2 or both. In another preferred embodiment, the photo-thermal conversion materials of the transparent photo-thermal conversion films 3a, 3b include a plurality of nano-particles of metal, as mentioned above.

With continued reference to FIGS. 1, 2 and 3, by way of example, the transparent photo-thermal conversion films 3a, 3b of another preferred embodiment have a perfect degree of transparency, with the transparent photo-thermal conversion films 3a, 3b further having a degree of scratch resistance, anti-chemical corrosion, anti-static or flexibility.

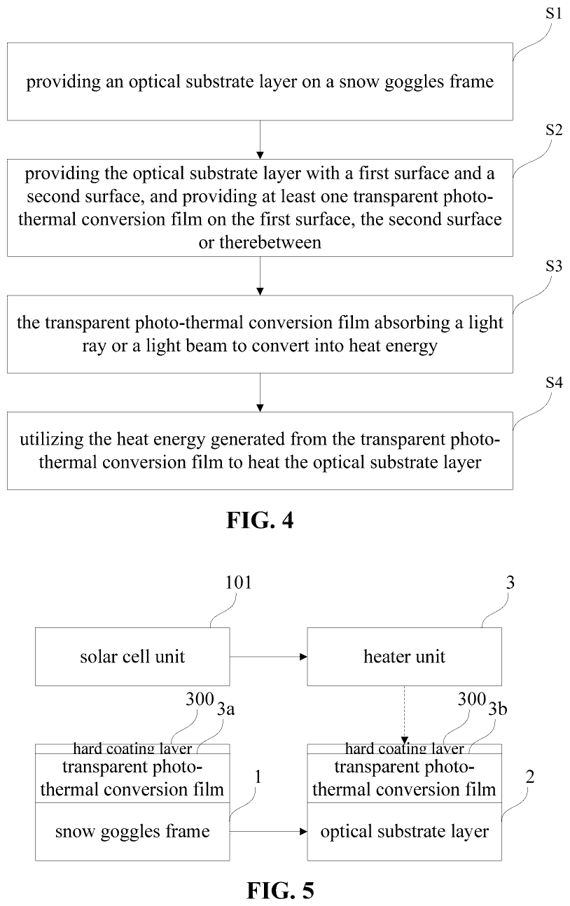

FIG. 4 shows a flow chart of a manufacturing method of snow goggles device in accordance with a preferred embodiment of the present invention. Turning now to FIGS. 1 through 4, by way of example, the manufacturing method of snow goggles device has a series of procedure (steps S1, S2, S3 and S4) which may be modified according to needs. The manufacturing method of snow goggles device of the present invention includes the step S1: providing the optical substrate layer 2 on the snow goggles frame 1 by means of assembling, molding, injection molding or other suitable manners, with preprocessing at least one predetermined surface (e.g. front side) of the snow goggles frame 1 to provide a pre-processed area thereon.

Still referring to FIGS. 1 through 4, by way of example, the manufacturing method of snow goggles device of the present invention further includes the step S2: providing the optical substrate layer 2 with the first surface 21 and the second surface 22 by means of polishing or other suitable manners, and providing at least one transparent photo-thermal conversion film 3b on at least one pre-processed area of the first surface 21, the second surface 22 or therebetween by means of sputtering, laser printing, spray coating, shower coating, adhering or other suitable manners.

Referring back to FIGS. 1 through 3, by way of example, at least one transparent photo-thermal conversion film 3a of another preferred embodiment is selected from a transparent film, a semi-transparent film or an opaque film, and is provided on at least one pre-processed area (i.e., outer surface, inner surface or other suitable surfaces) of the snow goggles frame 1 by sputtering, laser printing, spray coating, shower coating, adhering or other suitable manners.

Referring now to FIGS. 1 through 4, by way of example, the manufacturing method of snow goggles device of the present invention further includes the step S3: one of the transparent photo-thermal conversion films 3a, 3b actively absorbing an ambient light ray or beam to convert into heat energy on the first surface 21 and the second surface 22 of the optical substrate layer 2 or the pre-processed area (i.e., outer surface, inner surface or other suitable surfaces) of the snow goggles frame 1.

Still referring to FIGS. 1 through 4, by way of example, the manufacturing method of snow goggles device of the present invention further includes the step S4: utilizing the heat energy actively generated from the transparent photo-thermal conversion films 3a, 3b to heat the optical substrate layer 2 for preventing the optical substrate layer 2 (or snow goggles frame 1) turning into a frozen state so as to provide a characteristic of anti-frozen function, anti-frosted function or anti-fog function.

FIG. 5 shows a block diagram of a snow goggles device having an actively defrosting thin film in accordance with another preferred embodiment of the present invention. Turning now to FIG. 5, by way of example, the snow goggles device of the preferred embodiment includes a snow goggles frame 1, at least one solar cell unit 101, an optical substrate layer 2, at least one heater unit 3, a plurality of transparent photo-thermal conversion films 3a, 3b and a plurality of hard coating layers 300 selectively formed on the transparent photo-thermal conversion films 3a, 3b.

With continued reference to FIG. 5, by way of example, in addition to the transparent photo-thermal conversion films 3a, 3b, the solar cell unit 101 electrically connects with the heater unit 3 which can heat the snow goggles frame 1 or the optical substrate layer 2. In another preferred embodiment, the at least one solar cell unit 101 is integrally formed on a predetermined position (e.g., outer surface) of the snow goggles frame 1.

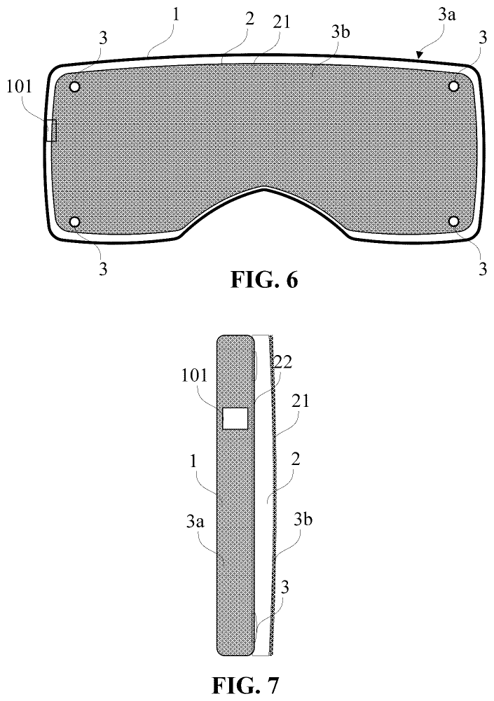

FIG. 6 shows a schematic front view of a snow goggles device having an actively defrosting thin film in accordance with a second preferred embodiment of the present invention. FIG. 7 shows a schematic side view of a snow goggles device having the actively defrosting thin film in accordance with the second preferred embodiment of the present invention, corresponding to the snow goggles device shown in FIG. 6.

Referring now to FIGS. 5, 6 and 7, by way of example, the at least one heater unit 3 of another preferred embodiment is selected from a frame-heating member, a heating wire member, a transparent heating film member, with the at least one heater unit 3 being also selected from a PTC (positive temperature coefficient) thermistor or other equivalent component.

With continued reference to FIGS. 5, 6 and 7, by way of example, the least one solar cell unit 101 of another preferred embodiment electrically connects with a switch button unit, a press key switch unit, a touch-panel switch unit, a tag switch unit or other equivalent switch units.

With continued reference to FIGS. 5, 6 and 7, by way of example, the least one solar cell unit 101 of another preferred embodiment further electrically connects with a lithium-battery unit, a button cell battery unit or other equivalent auxiliary power supply units which are provided on the snow goggles frame 1. In another preferred embodiment, the at least one solar cell unit 101 further electrically connects with a primary cell unit, a secondary cell unit or other equivalent auxiliary power supply units.

With continued reference to FIGS. 5, 6 and 7, by way of example, the least one heater unit 3 of another preferred embodiment selectively engages with a predetermined portion of the first surface 21 and the second surface 22 of the optical substrate layer 2 or therebetween. Provided between the first surface 21 and the second surface 22 of the optical substrate layer 2 is a side peripheral surface to engage with the at least one heater unit 3. In another preferred embodiment, the at least one heater unit 3 engages with the side peripheral surface between the first surface 21 and the second surface 22 which is preferably provided with a groove to receive the at least one heater unit 3.

With continued reference to FIGS. 5, 6 and 7, by way of example, the least one heater unit 3 of another embodiment is integrally formed on a predetermined portion of the snow goggles frame 1, including outer surfaces, inner surfaces or others, to form a protruded or recessed portion thereon.

With continued reference to FIGS. 5, 6 and 7, by way of example, the least one heater unit 3 of another preferred embodiment includes at least one illumination member (not shown) electrically connecting with the at least one solar cell unit 101, with the at least one illumination member selecting from an emergency LED element, a solar-cell illumination element, a florescent reflection element or combinations thereof. The illumination member further electrically connects with a switch unit (not shown).

Still referring to FIGS. 5, 6 and 7, by way of example, in comparison with the first embodiment, the manufacturing method of snow goggles device of the second preferred embodiment of the present invention further includes the step: providing the optical substrate layer 2 on a front side of the snow goggles frame 1 by means of assembling, molding, injection molding or other suitable manners.

With continued reference to FIGS. 5, 6 and 7, by way of example, the manufacturing method of snow goggles device of the second preferred embodiment of the present invention further includes the step: providing the optical substrate layer 2 with the first surface 21 and the second surface 22, and providing the at least one heater unit 3 on at least one pre-processed area of the first surface 21, the second surface 22 or a side surface therebetween.

With continued reference to FIGS. 5, 6 and 7, by way of example, the manufacturing method of snow goggles device of the second preferred embodiment of the present invention further includes the step: electrically connecting the at least one heater unit 3 with the at least one solar cell unit 101 by means of molding or other processing manners, with the at least one solar cell unit 101 and switch unit thereof provided on the pre-processed area (i.e., left side, right side or both) of the snow goggles frame 1.

With continued reference to FIGS. 5, 6 and 7, by way of example, the manufacturing method of snow goggles device of the second preferred embodiment of the present invention further includes the step: manually, automatically or other manners operating the switch unit to switch on or off a power supply unit so that the power supply unit can further supply an electric current to the at least one heater unit 3 to suitably heat the optical substrate layer for preventing the optical substrate layer (or the snow goggles frame 1) turning into a frozen state so as to provide a characteristic of anti-frozen function, anti-frosted function or anti-fog function.

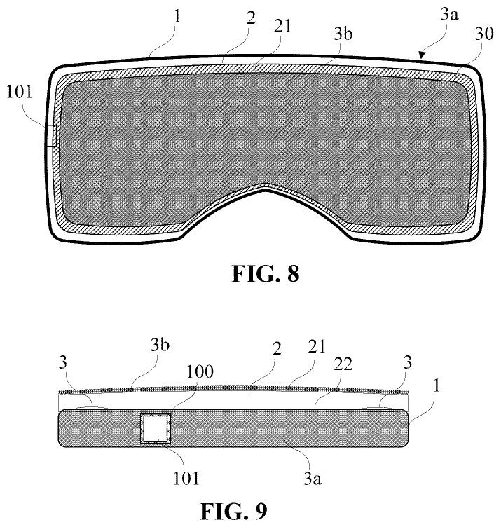

FIG. 8 is a schematic front view of a snow goggles device having an actively defrosting thin film in accordance with a third preferred embodiment of the present invention corresponding to the snow goggles device shown in FIGS. 6 and 7. Turning now to FIG. 8, by way of example, in comparison with the second embodiment, the snow goggles device of the second preferred embodiment of the present invention includes a snow goggles frame 1, at least one solar cell unit 101, an optical substrate layer 2, at least one frame-heating unit 30 and a plurality of transparent photo-thermal conversion films (ultra-thin films) 3a, 3b.

Still referring to FIG. 8, by way of example, the frame-heating unit 30 is provided to surround a predetermined periphery of the optical substrate layer 2, with preferably engaging with a predetermined portion between the first surface 21 and the second surface 22, with the frame-heating unit 30 selecting from a frame-heating member, a heating wire member, a transparent heating member (i.e., thin film) or combinations thereof.

FIG. 9 is a schematic side view of a snow goggles device having the actively defrosting thin film in accordance with a fourth preferred embodiment of the present invention. Turning now to FIG. 9, by way of example, in comparison with the second embodiment, the snow goggles device of the fourth preferred embodiment includes a snow goggles frame 1, at least one compartment 100 and at least one lid (not shown) in which to contain at least one solar cell unit 101 (or battery) and a switch unit thereof.

Still referring to FIG. 9, by way of example, in another embodiment, the solar cell unit 101 and the switch unit are coated by a layer of strengthening material in and around the compartment 100 for reinforcing entire structure.

Although the invention has been described in detail with reference to its presently preferred embodiment, it will be understood by one of ordinary skills in the art that various modifications can be made without departing from the spirit and the scope of the invention, as set forth in the appended claims.

What is claimed is:

1. A snow goggles device comprising:
an optical substrate layer provided with a first surface and a second surface;
a snow goggles frame provided with a predetermined area, with providing the optical substrate layer on the predetermined area of the snow goggles frame; and
at least one transparent photo-thermal conversion film provided on the first surface, the second surface or therebetween;
wherein the transparent photo-thermal conversion film can absorb a light ray or a light beam to convert into heat energy which can be utilized to heat the optical substrate layer for preventing the optical substrate layer turning into a frozen state.

2. The snow goggles device as defined in claim 1, wherein the transparent photo-thermal conversion film formed on the optical substrate layer further electrically connects with at least one solar cell unit.

3. The snow goggles device as defined in claim 1, wherein the transparent photo-thermal conversion film is integrally formed on the snow goggles frame, with the transparent photo-thermal conversion film further electrically connects with at least one solar cell unit.

4. The snow goggles device as defined in claim 1, wherein the transparent photo-thermal conversion film is made of a photo-thermal conversion material or a mixture of photo-thermal conversion material and thermochromic dye material.

5. The snow goggles device as defined in claim 4, wherein the photo-thermal conversion material is selected from an infrared-thermal conversion material.

6. The snow goggles device as defined in claim 4, wherein the infrared thermal conversion material includes a plurality of tungsten oxide micro-particles, a plurality of composite tungsten oxide micro-particles, a plurality of titanium dioxide micro-particles, a plurality of composite titanium dioxide micro-particles, a plurality of titanium dioxide/antimony-doped tin oxide (TiO2@ATO) core-shell micro-particles and combinations thereof.

7. The snow goggles device as defined in claim 1, wherein the transparent photo-thermal conversion film is further formed with at least one hard coating layer.

8. A manufacturing method of a snow goggles device comprising:
providing an optical substrate layer on a snow goggles frame;
providing the optical substrate layer with a first surface and a second surface and providing at least one transparent photo-thermal conversion film on the first surface, the second surface or therebetween;
the transparent photo-thermal conversion film absorbing a light ray or a light beam to convert into heat energy; and
utilizing the heat energy generated from the transparent photo-thermal conversion film to heat the optical substrate layer for preventing the optical substrate layer turning into a frozen state.

9. The method as defined in claim 8, wherein the transparent photo-thermal conversion film formed on the optical substrate layer further electrically connects with at least one solar cell unit.

10. The method as defined in claim 8, wherein the transparent photo-thermal conversion film is integrally formed on the snow goggles frame, with the transparent photo-thermal conversion film further electrically connects with at least one solar cell unit.

11. The method as defined in claim 8, wherein the transparent photo-thermal conversion film is made of a photo-thermal conversion material or a mixture of photo-thermal conversion material and thermochromic dye material.

12. The method as defined in claim 11, wherein the photo-thermal conversion material is selected from an infrared-thermal conversion material which is selectively added in the snow goggles frame, the optical substrate layer or both.

13. The method as defined in claim 11, wherein the infrared-thermal conversion material includes a plurality of tungsten oxide micro-particles, a plurality of composite tungsten oxide micro-particles, a plurality of titanium dioxide micro-particles, a plurality of composite titanium dioxide micro-particles, a plurality of titanium dioxide/antimony-doped tin oxide (TiO2@ATO) core-shell micro-particles and combinations thereof.

14. The method as defined in claim 8, wherein the transparent photo-thermal conversion film is further formed with at least one hard coating layer.
